(19) United States
(12) Patent Application Publication
(10) Pub. No.: US 2011/0093058 A1
(43) Pub. Date: Apr. 21, 2011

(54) GRAFT INCLUDING EXPANDABLE MATERIALS

(76) Inventor: Gil Vardi, Town and Country, MO (US)

(21) Appl. No.: 12/531,209

(22) PCT Filed: Mar. 12, 2008
(86) PCT No.: PCT/US08/56652

§ 371 Date: May 6, 2010

(30) Foreign Application Priority Data
Mar. 12, 2007 (US) 11/717485

(51) Int. Cl. A61F 2/82

(52) U.S. Cl. 623/1.15

(57) ABSTRACT

A graft for facilitating treatment of a deformity in a blood vessel wall includes a tubular body defining a first end and an opposing second end. At least a portion of the tubular body includes a super-absorbent material integrated into the tubular body and configured to expand upon exposure to moisture.

### CROSS REFERENCE TO RELATED APPLICATIONS

This application claims priority from U.S. patent application Ser. No. 11/717,485 to Vardi, filed on Mar. 12, 2007 and published as US Patent Publication Number 2007/0179600 on Aug. 2, 2007.

### BACKGROUND OF THE INVENTION

This invention relates generally to treatment of a deformity in a blood vessel and, more particularly, to methods and apparatus for treating a deformity, such as an aneurysm, in a blood vessel wall.

Stent grafts may be used to treat aneurysms in a patient's vascular system. An aneurysm is a degeneration of a blood vessel wall whereby the wall may weaken and balloon outwardly. Left untreated, an aneurysm may rupture causing fatal hemorrhaging. Conventional stent grafts typically include a stent forming an elongated tubular wire frame that provides structural support for the vessel wall and a tubular graft positioned about the wire frame to facilitate blood flow through the blood vessel while preventing blood flow into the aneurysm.

The traditional method of treating an aneurysm within a large vessel, such as an abdominal aortic aneurysm, includes an invasive surgical repair procedure. The surgical procedure requires a significant abdominal incision so that the stent graft may be implanted directly into the affected area. The patient is placed under general anesthesia and requires a significant amount of time in an intensive care unit following the procedure for post-operative recovery.

Due to the complexities of surgical repair, alternative approaches have been developed to deploy a stent graft endoluminally. Past approaches have included the introduction of multiple stent grafts that are expandable by a balloon catheter or are self-expanding. In addition, single stent grafts have been employed that include multiple branches. A problem with the existing stent graft configurations is the difficulty of treating aneurysms located near a bifurcation in the vasculature. Another problem is the insertion of devices designed to fit within the aorta, which requires a surgical incision due to the large profile of such devices.

### SUMMARY OF THE INVENTION

According to one embodiment of the present invention, there is provided a graft having a substantially tubular body with a first end and an opposing second end. The tubular body is comprised of a substantially flexible graft material. The graft further includes a super-absorbent material within the substantially tubular body, the super-absorbent material having an initial dry volume and configured to absorb moisture so as to form a swollen material having a volume of at least twice the initial dry volume.

According to another embodiment of the present invention, there is provided a stent graft, including a graft as described above and further including a stent positioned with respect to the graft, the stent comprising a support structure to facilitate retaining the stent graft with respect to the deformity.

According to yet another embodiment of the present invention, there is provided a method for treating a deformity in a blood vessel wall. The method includes introducing a graft through an access site, the graft comprising a super-absorbent material having an initial dry volume and capable of expanding to a swollen volume which is at least two times the initial dry volume, advancing the graft until at least a portion of the graft extends across the deformity, and exposing the super-absorbent material to moisture, thereby expanding the super-absorbent material to the swollen volume, wherein the swollen volume is sufficient to fill a cross-sectional area between the graft and the blood vessel wall.

Unless otherwise defined, all technical and scientific terms used herein have the same meaning as commonly understood by one of ordinary skill in the art to which this invention belongs. Although methods and materials similar or equivalent to those described herein can be used in the practice or testing of the embodiments of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

FIG. 1 is a plan view of an exemplary stent graft assembly;

FIG. 2 is a cross-sectional view of the stent graft assembly taken along line 1-1 of FIG. 1;

FIG. 3 shows a partial cross-sectional view of an aneurysm in the process of being repaired in accordance with one embodiment;

FIG. 4 is a schematic view of a stent graft including a perforated inflation tube;

FIG. 5 is a schematic view of a stent graft including an inflation port;

FIG. 6 is a schematic view of a stent graft including a sponge material;

FIG. 7 is a schematic view of a stent graft including two expandable cuffs;

FIG. 8 is a schematic view of a stent graft including a cuff that extends substantially the entire length of the graft;

FIG. 9 is a schematic view of a stent graft that includes four cuffs attached to the graft;

FIG. 10 is a plan view of an alternative exemplary stent graft assembly;

FIG. 11 is a cross-sectional view of the stent graft assembly taken along line 2-2 of FIG. 10;

FIG. 12 is a perspective sectional view of an alternative exemplary stent graft in an initially compressed configuration;

FIG. 13 is an end view of the stent graft shown in FIG. 12 in an expanded configuration;

FIG. 14 is a perspective sectional view of an alternative exemplary graft in an initially compressed configuration;

FIG. 15 is a cross-sectional view of the graft of FIG. 14 in an expanded configuration;

FIG. 16 is a perspective sectional view of an alternative exemplary graft in an initially compressed configuration;

FIG. 17 is a perspective sectional view of the graft of FIG. 16 in an expanded configuration;

FIG. 18 is a perspective sectional view of the graft of FIGS. 16 and 17 in an alternative configuration, shown in an initially compressed configuration; and

FIG. 19 is a perspective sectional view of the graft of FIG. 18 in an expanded configuration.

### DETAILED DESCRIPTION OF THE INVENTION

Exemplary embodiments of stent grafts are described below. In one embodiment, a stent graft assembly includes at least one stent graft having an expandable cuff at one end. A second stent graft may be employed at the same location to accommodate a branched artery or a larger size than can be percutaneously inserted. In one embodiment, the cuff is inflatable, while in an alternative embodiment, the cuff includes a sponge material that expands upon exposure to moisture. In a further embodiment, the stent graft includes a first cuff located at the distal end and a second cuff located at the proximal end. In a further embodiment, each stent graft has a flattened side when the stent grafts are placed within a vessel.

The methods and apparatus for a stent graft described herein are illustrated with reference to the figures wherein similar numbers indicate the same elements in all figures. Such figures are intended to be illustrative rather than limiting and are included herewith to facilitate explanation of exemplary embodiments of the stent graft.

The terms “distal” and “proximal” as used herein refer to the orientation of the stent graft within the body of a patient. As used herein, “distal” refers to that end of the stent graft extended farthest into the body while “proximal” refers to that end of the stent graft located farthest from the distal end of the stent graft.

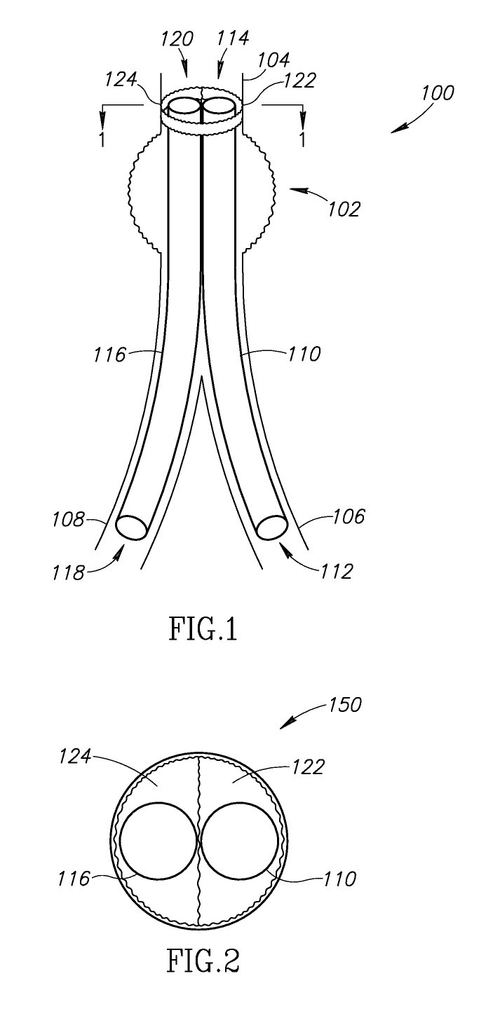

FIG. 1 shows a plan view of a stent graft assembly 100. In the exemplary embodiment, an aneurysm 102 is an abdominal aortic aneurysm in an aorta 104 that has common iliac arteries 106 and 108. The invention is not limited to the repair of abdominal aortic aneurysms. For example, the invention may be used in the thoracic aorta to repair thoracic aortic aneurysms. Furthermore, the invention may be used in a variety of body lumen (either bifurcated or non-bifurcated) where stent grafts are inserted.

In the exemplary embodiment, a first stent graft 110 includes a proximal end 112 and a distal end 114 and a second stent graft 116 includes a proximal end 118 and a distal end 120. An expandable cuff 122 is attached to distal end 114 of stent graft 110 and an expandable cuff 124 is attached to distal end 120 of stent graft 116. Stent grafts 110 and 116 have a generally circular cross-sectional configuration. Cuffs 122, 124 may be expanded with a fluid and inflated to a specific expanded configuration. Alternatively, cuffs 122, 124 may comprise a sponge material that expands upon exposure to moisture. In one embodiment, cuffs 122, 124 have a “D” shape in the expanded configuration. Alternatively, cuffs 122, 124 have a substantially spherical or cylindrical shape in the expanded configuration, but due to the pressure applied to the adjacent cuff, each cuff conforms to a “D” shape when expanded in the vessel due to space constraints.

FIG. 2 is a cross-sectional view 150 taken along line 1-1 of FIG. 1. Expandable cuffs 122 and 124 are shown in their expanded state. Each of cuffs 122, 124 have a “D” configuration when expanded while the cross-sections of stent grafts 110 and 116 remain substantially circular.

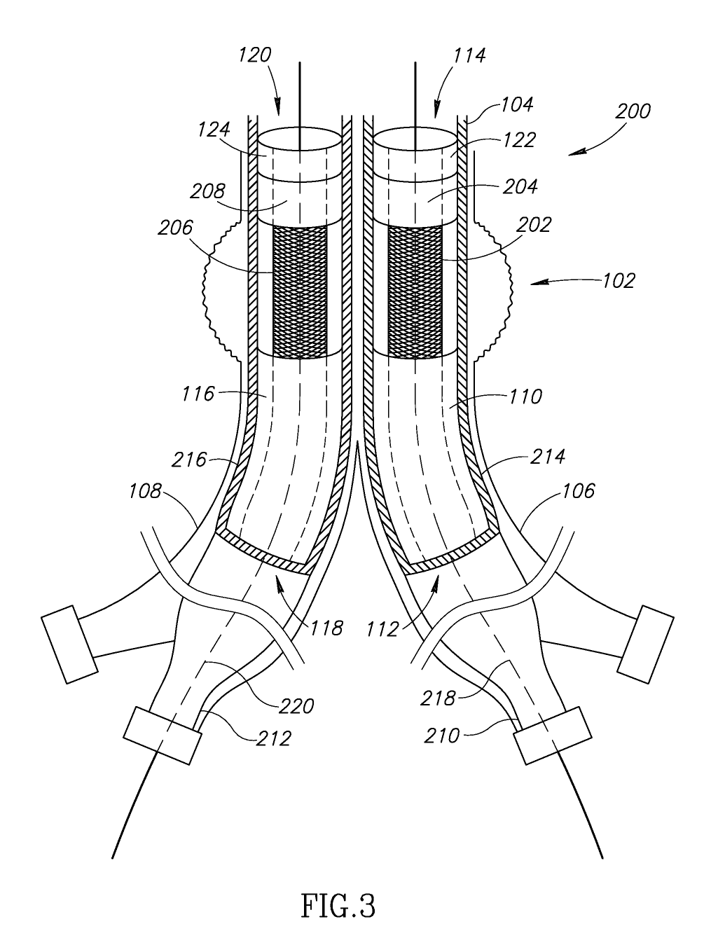

FIG. 3 shows a partial cross-sectional view of aneurysm 102 in the process of being repaired by stent graft assembly 200. Stent graft 110 is a composite device including a stent 202 and a graft 204, and stent graft 116 is a composite device including a stent 206 and a graft 208.

Stents 202 and 206 are elongated tubular wire frame devices manufactured from one or more of a variety of materials providing sufficient structural support and biocompatibility to allow for the treatment of a weakened or diseased vessel wall. Examples of suitable materials include stainless steel and nitinol. Grafts 204 and 208 are elongated tubular devices through which blood may flow. Grafts 204 and 208 are manufactured from one or more of a variety of materials providing sufficient mechanical properties for allowing the flow of blood and biocompatibility. Examples of suitable materials include DACRON® materials (polyethylene terephthalate) and TEFLON® materials (polytetrafluoroethylene).

In one embodiment, inflatable cuffs 122 and 124 are manufactured from one or more of a variety of materials allowing for a radially outward force to be exerted against the other of the cuffs and a vessel wall. A suitable material for the fabrication of inflatable cuffs 122 and 124 include a compliant material such as latex. An alternative material for the fabrication of inflatable cuffs 122 and 124 include a non-compliant material such as nylon.

In one embodiment, expandable cuffs 122, 124 are fabricated from a sponge material. The material is at least one of a natural sponge material and a synthetic absorbent material that functions as a sponge. In the example embodiment, the sponge material includes a thrombogenic material. For example, the sponge material is soaked with a pro-coagulant. Upon exposure to moisture, e.g., the patient's blood, the moisture is absorbed by the sponge material, causing the cuff to expand. The blood reacts with the thrombogenic material and causes the blood to clot in the expanded cuff and harden in the expanded shape.

In the example embodiment, stent graft 110 and stent graft 116 are delivered by catheters. A first introducer delivery device 210 and a second introducer delivery device 212, both including a tubular sheath, are inserted into the patient's vasculature through the femoral artery by means of a femoral arteriotomy or percutaneous delivery. First delivery catheter 214 and second delivery catheter 216 are then fed into the vasculature by means of these introducers. A first guide wire 218 is advanced through the femoral artery, external iliac artery, common iliac artery 106, and aneurysm 102 until it extends into aorta 104. A second guide wire 220 is advanced through the femoral artery, external iliac artery, common iliac artery 108, and aneurysm 102 until it also extends into aorta 104. First delivery catheter 214 and second delivery catheter 216 are guided by means of first guide wire 218 and second guide wire 220 until each extend across aneurysm 102.

Stent graft 110 is introduced using first delivery catheter 214 and stent graft 116 is introduced using second delivery catheter 216 until at least a portion of distal end 114 of stent graft 110 and distal end 120 of stent graft 116 extend across aneurysm 102 and are aligned with each other. In one embodiment, the alignment of stent grafts 110 and 116 is monitored with the use of radio-opaque markers.

Cuff 122 is expanded to exert a radially outward force against cuff 124 and the vessel wall. Cuff 124 is expanded to exert a radially outward force against cuff 122 and the vessel wall. Cuffs 122 and 124 may be expanded either simultaneously or sequentially. In one embodiment, cuffs 122 and 124 are inflated with a variety of materials that promote a seal between inflatable cuffs 122, 124 and the vessel wall. In one example, inflatable cuffs 122 and 124 are inflated with a hardening agent, such as collagen or a mixture of thrombin and the patient's blood. After inflation, the material hardens and the cuff maintains its expanded shape even if the integrity of the cuff is compromised. In another example, inflatable cuffs 122, 124 are inflated with a synthetic material such as an epoxy that hardens upon inflation of cuffs 122, 124 and maintains the expanded cuff shape even if the integrity of the cuff is compromised. In either example, cuffs 122, 124 are inflated to form a seal between the stent graft and the vessel wall even if the integrity of a cuff is compromised. In another embodiment, inflatable cuffs 122 and 124 are inflated with a saline solution, allowing for easy deflation and retrieval of stent graft 110. At the completion of the delivery procedure, the delivery devices are removed and any incisions are closed by known techniques such as applying pressure to stop the bleeding, suturing by standard vascular surgical techniques, and utilizing a known closure device.

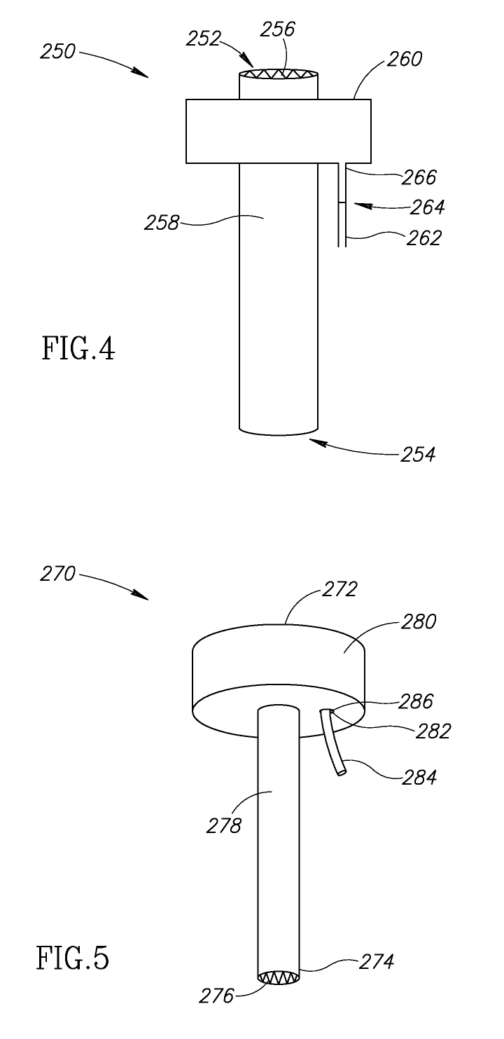

FIG. 4 is a schematic view of a stent graft 250 including a distal end 252, a proximal end 254, a stent 256, a graft 258 and a cuff 260. An inflation tube 262 extends from cuff 260 and is used to provide inflation fluid to cuff 260. In one embodiment, inflation tube 262 includes a weakened section 264 or a closure device near cuff end 266. Weakened section 264 is, in one embodiment, a perforated section configured to sever and allow inflation tube 262 to separate. Weakened section 264 is configured to provide a release mechanism of inflation tube 262 from cuff 260. Weakened section 264 has sufficient strength to enable tube 262 to provide enough fluid to cuff 260 such that cuff 260 inflates to the desired size and shape. In addition, weakened section 264 is configured to sever when a sufficient stress is applied to tube 262. Such stress is applied after cuff 260 has been adequately inflated and as tube 262 is pulled away from stent graft 250. In one embodiment, this stress is a pressure less than 5 atmospheres. In another embodiment, this stress is a pressure of about 1-2 atmospheres. In one embodiment, tube 262 is attached to a delivery mechanism, such as a delivery catheter, and when the delivery catheter is removed inflation tube 262 is severed at weakened section 264. In the exemplary embodiment, tube 262 is severed after cuff 260 is inflated and hardened such that cuff 260 retains its expanded configuration even upon severance of tube 262 and hence the loss of integrity of cuff 260.

FIG. 5 illustrates an alternative embodiment of a stent graft 270 including a distal end 272, a proximal end 274, a stent 276, a graft 278 and a cuff 280. Cuff 280 includes an inflation port 282 configured to accept and release an inflation tube 284. In one embodiment, inflation port 282 includes a valve 284 configured to prevent fluid to flow out of cuff 280 after cuff 280 is inflated and tube 284 is removed from inflation port 282. In the exemplary embodiment, valve 286 is a flap valve in which the flap is a compliant member, although other types of valves can be used as long as they provide a seal sufficient to maintain cuff 280 in the expanded configuration. Valve 286 is configured to remain in the sealed position after removal of tube 284. In the exemplary embodiment, tube 284 is inserted within valve 284 prior to insertion of stent graft 270 into the body. After appropriate positioning and expansion of stent graft 270 within a vessel, cuff 280 is inflated with a fluid that passes through inflation tube 284. The delivery catheter is then removed along with inflation tube 284. Upon removal of inflation tube from inflation port 282, valve 286 seals and prevents fluid from escaping from expanded cuff 280. Alternatively, inflation tube 284 remains within inflation port 282 until the inflation media within cuff 280 hardens such that cuff 280 remains in the expanded configuration. Inflation tube 284 is then removed from inflation port 282 without the contents within cuff 280 escaping into the lumen.

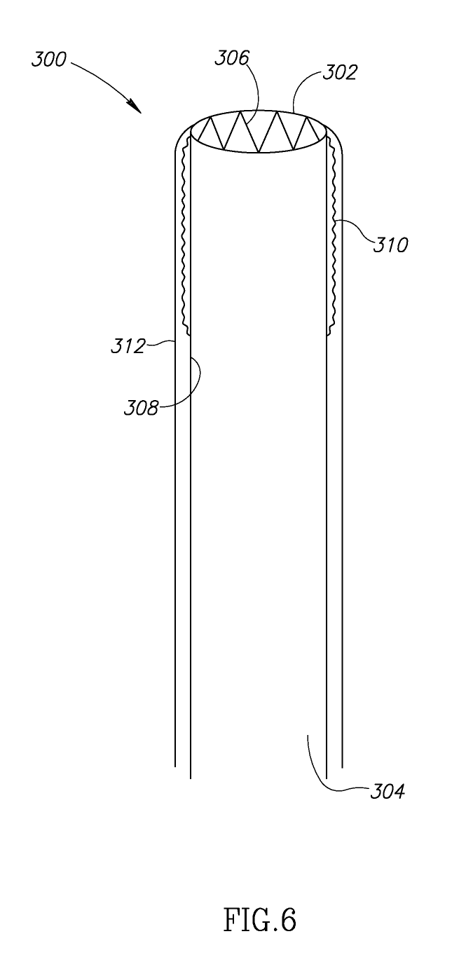

FIG. 6 is a schematic view of a stent graft 300 including a distal end 302, a proximal end 304, a stent 306, a graft 308 and a cuff 310. Cuff 310 is fabricated from a sponge material that expands upon absorption of liquid. Accordingly, during insertion of stent graft 300 into a body, cuff 310 is covered with a shield 312. After stent graft 300 is located at the appropriate location, shield 310 is removed and the sponge material of cuff 310 is exposed to the patient's blood. In one embodiment, shield 312 is a porous structure. In another embodiment, shield 312 is a non-porous structure.

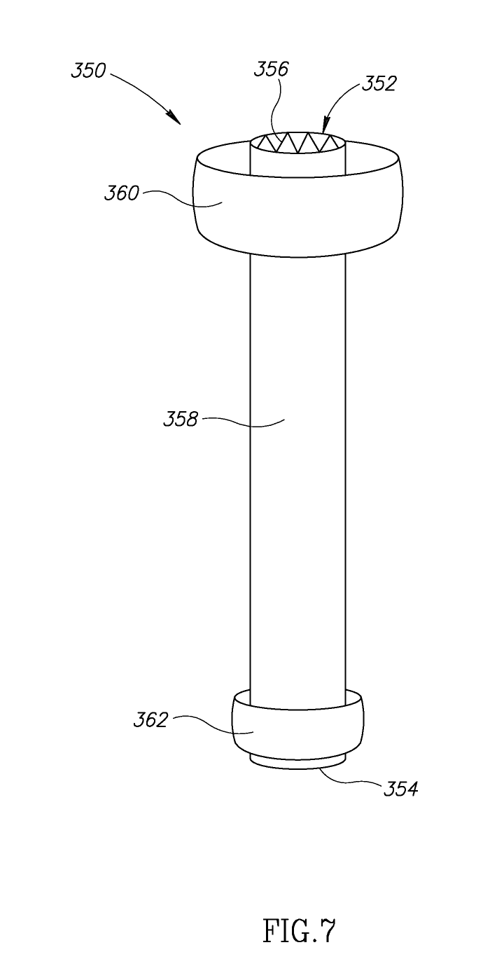

FIG. 7 illustrates a stent graft 350 including a distal end 352, a proximal end 354, a stent 356, a graft 358, a distal cuff 360, and a proximal cuff 362. Distal cuff 360 is configured to seal a large lumen, such as the aorta, either alone or in combination with a second stent graft 350 and proximal cuff 362 is configured to seal a smaller lumen, such as a common iliac artery. In one embodiment, cuffs 360, 362 are inflatable cuffs and use at least one of an inflation port with a valve and a severable inflation tube. In another embodiment, cuffs 360, 362 are fabricated from a sponge material that expands upon exposure to moisture. In a further embodiment, one of cuffs 360, 362 is an inflatable cuff while the other of cuffs 360, 362 is fabricated from a sponge material.

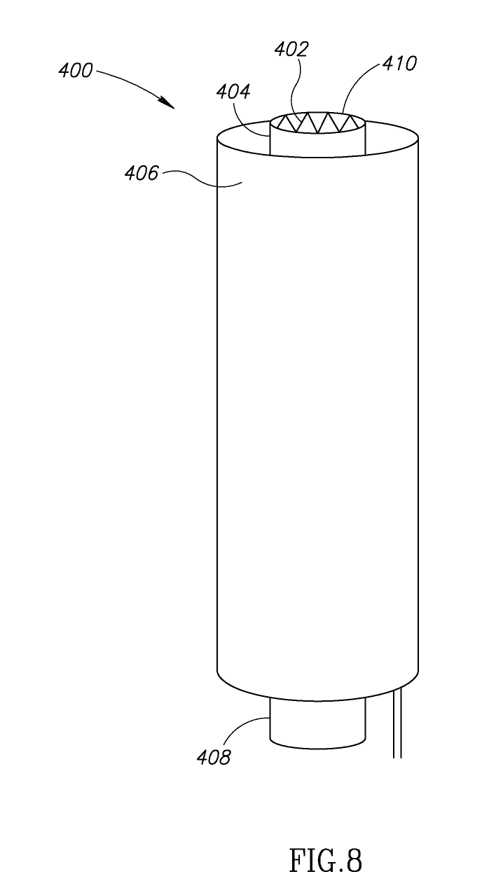

FIG. 8 illustrates a stent graft 400 including a stent 402, a graft 404 and a cuff 406. Graft 400 includes a proximal end 408 and a distal end 410. Cuff 406 extends substantially the entire length of stent graft 400. In one embodiment, cuff 406 extends from within half an inch of proximal end 408 to within half an inch of distal end 410. Cuff 406 includes an inflation tube 408 used to inflate cuff 406 with a fluid.

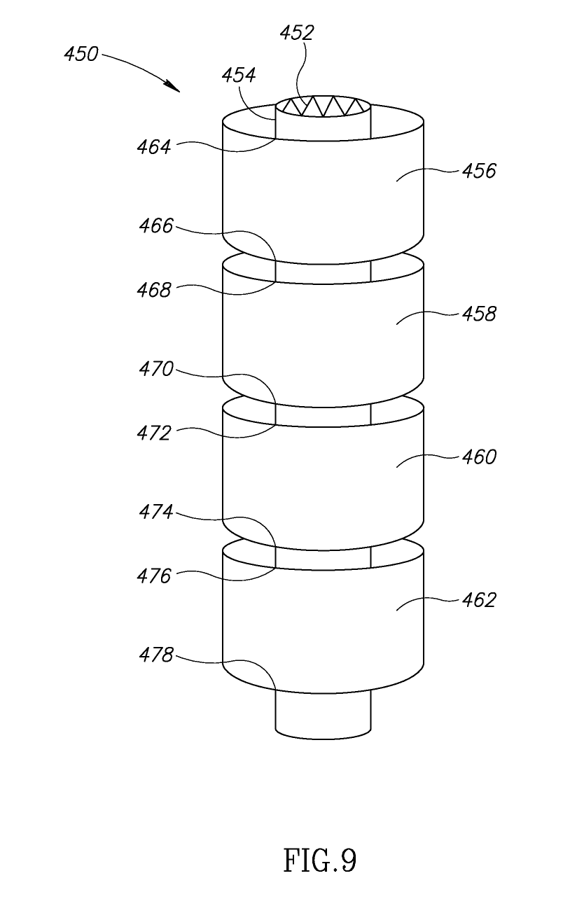

FIG. 9 illustrates a stent graft 450 including a stent 452, a graft 454, a first cuff 456, a second cuff 458, a third cuff 460, and a fourth cuff 462. First cuff 456 includes a distal end 464 and a proximal end 466 and cuff 456 is attached to graft 454 at distal end 464. Second cuff 458 includes a distal end 468 and a proximal end 470 and cuff 458 is attached to graft 454 at distal end 470. Third cuff 460 includes a distal end 472 and a proximal end 474 and cuff 460 is attached to graft 454 at distal end 472. Fourth cuff 462 includes a distal end 476 and a proximal end 478 and cuff 462 is attached to graft 454 at distal end 476. In one embodiment, cuffs 456, 458, 460, and 462 are attached to graft 454 at only distal ends 464, 468, 472, and 476. In another embodiment, cuffs 456, 458, 460, and 462 are attached to graft 454 along their entire length. In a further embodiment, cuffs 456, 458, 460, and 462 are attached to graft 454 along a distal portion of cuffs 456, 458, 460, and 462 that extends to substantially a middle of each of cuffs 456, 458, 460, and 462 to form a skirt around graft 454 when cuffs 456, 458, 460, and 462 are expanded. In the embodiment shown in FIG. 9, cuffs 456, 458, 460, and 462 comprise a sponge material. Alternatively, cuffs 456, 458, 460, and 462 are inflatable members and an inflation tube extends between adjacent cuffs.

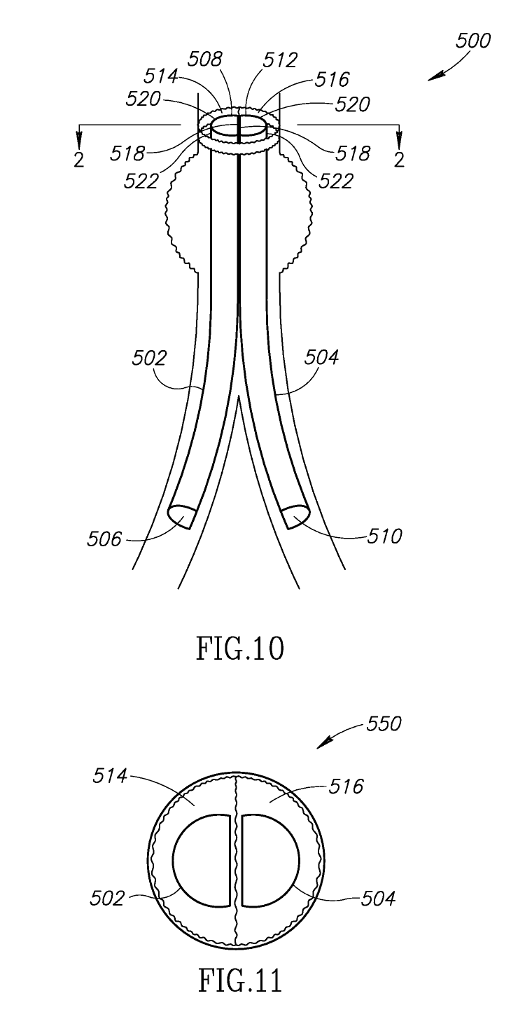

FIG. 10 is a plan view of a stent graft assembly 500 including a first stent graft 502 and a second stent graft 504. First stent graft 502 includes a proximal end 506 and a distal end 508 and second stent graft 504 includes a proximal end 510 and a distal end 512. An expandable cuff 514 is attached to distal end 508 of stent graft 502 and an expandable cuff 516 is attached to distal end 512 of stent graft 504. Stent grafts 502 and 504 have a generally “D” shaped cross-sectional configuration, for example a flattened or straight portion 518 attached to an arcuate or substantially semi-circular portion 520. Stent grafts 502 and 504 each include a radiopaque marker 522 attached to one side thereof. Radiopaque markers 522 are utilized to properly align stent grafts 502 and 504 during delivery such that flattened sides 518 are adjacent each other after stent grafts 502 and 504 have been inserted within the vessel.

Cuffs 514, 516 may be expanded with a fluid and inflated to a specific expanded configuration. Alternatively, cuffs 514, 516 may comprise a sponge material that expands upon exposure to moisture. Cuffs 514, 516 each have a “D” configuration (similar to the configuration of stent grafts 502, 504) when in the expanded configuration. Alternatively, cuffs 514, 516 have a substantially spherical or cylindrical shape in the expanded configuration, but due to the pressure applied to the adjacent cuff, each cuff conforms to a “D” shape when expanded in the vessel due to space constraints. In one embodiment, stent grafts 502 and 504 do not contact each other and a space extends between stent grafts 502 and 504 at distal ends 508 and 512. Cuffs 514 and 516 extend within the space and contact each other when stent grafts 502 and 504 are properly positioned within a vessel. In another embodiment, stent grafts 502 and 504 contact each other along flattened side 518 and cuffs 514 and 516 prevent fluid flowing between cuffs 502 and 504.

FIG. 11 is a cross-sectional view 550 taken along line 2-2 of FIG. 10. Expandable cuffs 514, 516 are shown in their expanded state. Each of cuffs 514, 516 have a “D” configuration when expanded and the cross sections of each of stent grafts 502, 504 also have a “D” configuration.

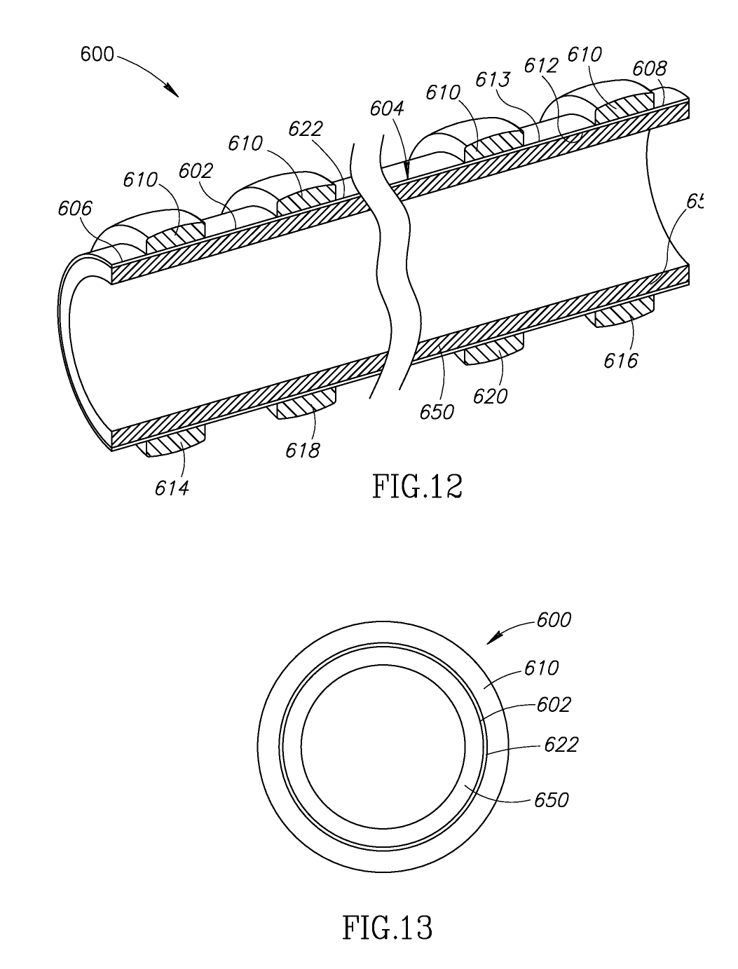

Referring to FIGS. 12 and 13, in one embodiment, a stent graft 600 includes a graft 602 for facilitating treatment of a deformity in a blood vessel wall. Graft 602 forms a tubular body 604 that defines a first end 606 and an opposing second end 608. It should be apparent to those skilled in the art and guided by the teachings herein provided that graft 602 may be made or fabricated using any suitable biocompatible material. In a particular embodiment, graft 602 is fabricated at least partially from a suitable polymeric material, such as a polyurethane and/or polymethane material.

In one embodiment, at least a portion of tubular body 604 includes a hydrogel material 610 configured to expand upon exposure to moisture. In a particular embodiment, hydrogel material 610 includes a suitable thrombogenic material and/or a suitable pro-coagulant material. Hydrogel material 610 may be in the form of a powder material, a gel material and/or at least one fiber. In one embodiment, as shown in FIG. 12, hydrogel material 610 is formed into a plurality of segmented portions, which extend along a length of stent graft 600. Alternatively, hydrogel material 610 extends along only a portion of the stent graft length or continuously along substantially an entire length of stent graft 600. In a particular embodiment, hydrogel material 610 is coated onto at least a portion of an inner surface 612 and/or at least a portion of an outer surface 613 of tubular body 604. Alternatively or in addition, at least one fiber (not shown) including hydrogel material 610 is coupled to or integrated with tubular body 604.

Referring further to FIG. 13, with stent graft 600 properly positioned within the vessel, hydrogel material 610 is configured to expand upon exposure to moisture. Stent graft 600 is guided through the vessel and properly positioned with stent graft 600, including hydrogel material 610, in an initially compressed or insertion configuration. Upon exposure to moisture within the vessel, e.g., exposure to blood, hydrogel material 610 expands radially outwardly to an expanded or deployed configuration such that hydrogel material 610 contacts and/or interferes with an inner surface of the vessel wall to facilitate sealingly retaining stent graft 600 properly positioned within the vessel. Additionally or alternatively, stent graft 600 expands from an initially compressed configuration to an expanded configuration. In a particular embodiment, hydrogel material 610 is configured to harden after expansion to the expanded configuration to further facilitate retaining stent graft 600 properly positioned within the vessel and/or to facilitate preventing endoleaks, i.e., leakage or passage of blood between outer surface 613 of stent graft 600 and the inner surface of the vessel wall, be forming a seal between the surfaces.

In a particular embodiment, a first cuff 614 is positioned at first end 606 and a second cuff 616 is positioned at second end 608. First cuff 614 and second cuff 616 include hydrogel material 610 and are configured to expand and exert a radially outward force against the blood vessel wall. Cuffs 614, 616 expand to seal a space or region between outer surface 613 of stent graft 600 and an inner surface of the vessel wall and retain stent graft 600 properly positioned within the blood vessel. In a further embodiment, upon expansion, first cuff 614 and/or second cuff 616 are configured to harden upon exposure to moisture, e.g., blood. In a further embodiment, additional cuffs, such as a third cuff 618 and/or a fourth cuff 620 are positioned about stent graft 600 to facilitate retaining stent graft 600 properly positioned within the blood vessel.

In an alternative embodiment, hydrogel material 610 is positioned between a first layer of material and a second layer of material. In a particular embodiment, tubular body 604 includes a first layer of material 622 and a second layer of material (not shown) that is coaxially positioned about first layer 622. Hydrogel material 610 is positioned between first layer 622 and the second layer. Upon expansion of stent graft 600 and/or expansion of hydrogel material 610, at least the second layer is moved radially outwardly such that the second layer contacts the inner surface of the vessel wall to facilitate sealingly positioning stent graft 600 within the vessel.

As shown in FIGS. 12 and 13, in the exemplary embodiment, stent graft 600 includes a stent 650 positioned with respect to graft 602. In one embodiment, graft 602 is coaxially positioned about at least a portion of an outer surface of stent 650. In alternative embodiments, graft 602 is positioned within stent 650 and configured to contact at least a portion of an inner surface of stent 650. Stent 650 includes a wire frame 652 that forms a support structure to facilitate retaining stent graft 600 with respect to the deformity. Stent 650, including wire frame 652, is fabricated of a biocompatible material including, without limitation, suitable metal materials, such as stainless steel, platinum, gold, titanium and nickel and/or composites or alloys thereof. In the exemplary embodiment, stent 650 is fabricated at least partially from a material having shape memory properties. Suitable materials include, without limitation, Nitinol and other known shape memory alloys (SMA) having properties that develop a shape memory effect (SME), which allows the material to return to an initial configuration after a force applied to the material to shape, stretch, compress and/or deform the material is removed. In a further embodiment, stent 650 is fabricated from a thermally treated metal alloy (TMA) including, without limitation, nickel titanium, beta titanium, copper nickel titanium and any combination thereof. In one embodiment, stent 650 is expandable using a balloon and/or another mechanism suitable for facilitating expanding stent 650. In an alternative embodiment, stent 650 is fabricated at least partially from a suitable polymeric material, such as a polyurethane and/or polymethane material. It should be apparent to those skilled in the art and guided by the teachings herein provided that stent 650 may be made or fabricated using any suitable biocompatible material preferably, but not necessarily, having suitable shape memory properties.

Further, stent 650 may have any suitable size, shape and/or configuration, which provide sufficient structural strength as required. In one embodiment, stent 650 is substantially shaped as a tube or cylinder to define support structure 652, as shown in FIGS. 12 and 13, defining a substantially circular cross-sectional area. Alternatively, stent 650 may define any suitable cross-sectional area, such as a polygonal cross-sectional area.

In one embodiment, stent 650 defines a first end and an opposing second end corresponding to first end 606 and second end 608 of graft 602, respectively. At least a portion of stent 650 includes hydrogel material 610, which is configured to expand upon exposure to moisture, such as by absorbing blood within the blood vessel. In one embodiment, hydrogel material 610 is formed about at least a portion of the wire or wires forming wire frame 652. In this embodiment, hydrogel material 610 is applied to wire frame 652 as a dry foam material. The dry foam hydrogel material 610 is configured to expand upon hydration. In a particular embodiment, hydrogel material 610 includes a suitable thrombogenic material and/or a suitable pro-coagulant material. Upon expansion, hydrogel material 610 is configured to harden.

Hydrogel material 610, in the form of a powder material, a gel material, a foam material and/or a fiber material, for example, is incorporated into and/or coupled to stent 650 and/or graft 602. In one embodiment, hydrogel material 610 is applied as a coating layer on at least a portion of stent 650 and/or graft 602 using a suitable method including, without limitation, a painting, spraying and/or dipping method. In an alternative embodiment, hydrogel material 610 is formed in a material sheet or layer that is coupled to stent 650 and/or graft 602 using a suture or other suitable coupling mechanism.

In a particular embodiment, hydrogel material 610 is coated onto at least a portion of stent 650, such as an inner surface and/or an outer surface of stent 650. In a further particular embodiment, hydrogel material 610 is formed in at least one fiber that is coupled to stent 650. For example, in one embodiment, the fiber (not shown), which includes hydrogel material 610, is wrapped about at least a portion of stent 650. Alternatively, hydrogel material 610 is integrated with stent 650. Similarly, hydrogel material 610 can be coupled to or integrated with graft 602 with or without coupling hydrogel material 610 or integrating hydrogel material 610 with stent 650. In an alternative embodiment, hydrogel material 610 is coupled to or integrated with stent 650 and configured to expand or swell such that hydrogel material 610 forms a graft component of stent graft 600. In this embodiment, hydrogel material 610 may be used in addition to or as an alternative to graft 602.

In one embodiment, a method is provided for treating a deformity in a blood vessel wall with stent graft 600 including graft 602 positioned about stent 650. Stent graft 600 is introduced through an access site. Stent graft 600 defines a first end and an opposing second end and includes hydrogel material 610 that is configured to expand upon exposure to moisture. Stent graft 600 is advanced through the blood vessel until at least a portion of stent graft 600 extends across the deformity. With stent graft 600 properly positioned within the blood vessel, hydrogel material 610 is configured to expand to form a seal between stent graft 600 and the blood vessel wall. In a particular embodiment, hydrogel material 610 is configured to harden upon expansion to facilitate retaining stent graft 600 properly positioned within the blood vessel and/or to facilitate preventing endoleaks from forming.

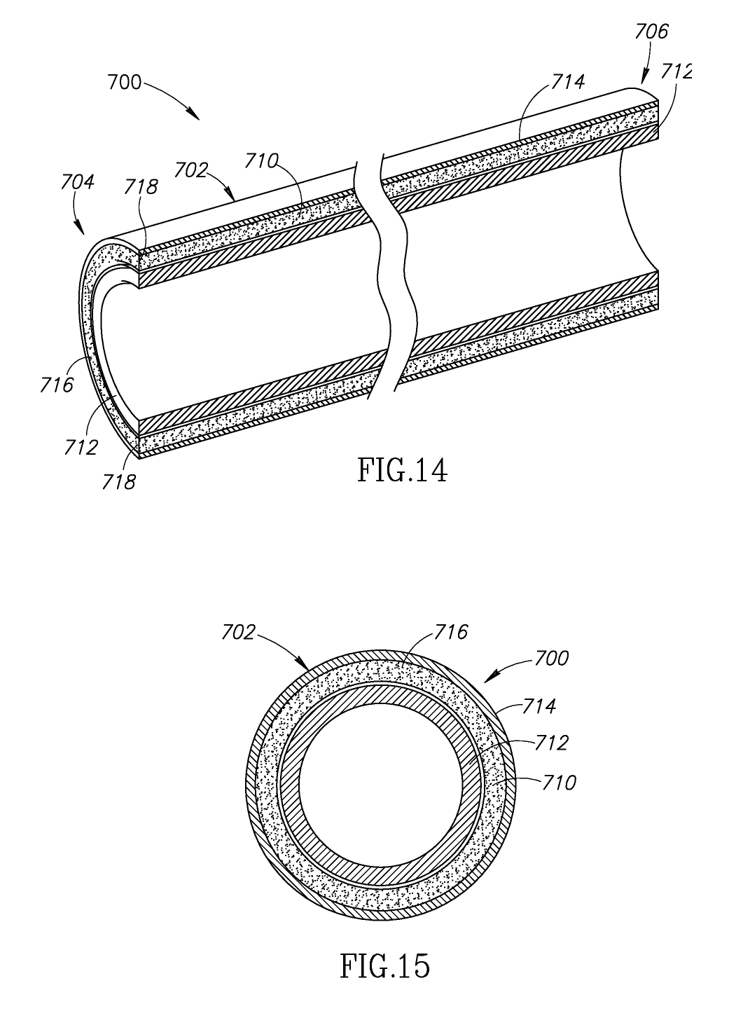

Reference is now made to FIG. 14, which is a perspective sectional view of an alternative exemplary graft 700 in an initially compressed configuration and to FIG. 15, which is a cross-sectional view of graft 700 of FIG. 14 in an expanded configuration. Graft 700 has a substantially tubular body 702 with a first end 704 and an opposing second end 706. Tubular body 702 is formed from a substantially flexible graft material. Graft 700 further includes a super-absorbent material 710 within tubular body 702. In one embodiment, the super-absorbent material 710 is integrated within tubular body 702. Super-absorbent material 710 is a material which is configured to absorb a large volume of moisture, thereby expanding the material. In some embodiments, super-absorbent material 710 may absorb an amount of moisture so as to result in a swollen volume which is at least twice its initial dry volume. In other embodiments, super-absorbent material 710 may absorb moisture of up to several thousand times its original weight, and in some instances, may change form. For example, a super-absorbent fiber may undergo such significant expansion that it can become a gel. Super-absorbent material 710 is shown in FIG. 14 with an initial dry volume, and shown in FIG. 15 with a swollen volume. Examples of super-absorbent materials include hydrogels, such as those described above, or other types of commercially available super-absorbent materials, such as ones available from Technical Absorbents Ltd. (Grimsby, UK). Super-absorbent material 710 may further include, but is not limited to, polymers, textiles, or other materials.

In some embodiments, a super-absorbent fiber may be manufactured by the following steps: A discontinuous fiber is coated with a binder material with the binder material adhering the fiber to one or more super absorbent particles. The binder may be present at an amount which is sufficient to substantially continuously coat the fibers. Plural coatings of various binder materials may be used. The binder material may be heat fusible or heat curable and the treated fibers mixed with other fibers for use in producing a wide variety of products. In other embodiments, the fiber itself may be comprised of super-absorbent material. The super-absorbent fiber may include polymers, such as polyacrylic acids or may include cellulose. Other polymers which may be used as a super-absorbent material include polyglycolic acid (PGA), polyurethane, polyvinyl alcohol (PVA), polyacrylamides, ethylene maleic anhydride copolymers, polyvinyl ethers, hydroxypropylcellulose, polyvinylmorpholinone, and polymers and copolymers of vinyl sulfonic acid, polyacrylates, polyacrylamides, polyvinyl amines, polyallylamines, polyvinylpyrridine. Other materials which have super-absorbent properties are, for example, acrylic fibers or other engineered polymers available from Lubrizol Corporation (Ohio, USA) and hydrogels available from Biocure, Inc. (Georgia, USA) for example. Additional materials which may have super-absorbent properties may include agar, algin, carrageenan, starch, pectin, guar gum, chitosan, and the like, modified natural materials such as carboxyalkyl cellulose, methyl cellulose, hydroxyalkyl cellulose, chitosan salt, dextran, and the like.

As shown in FIGS. 14 and 15, in one embodiment, super-absorbent material 710 is placed between two layers of graft material. In one embodiment, this may be done by co-extruding the materials. In other embodiments, a thin coating of super-absorbent material 710 may be applied to an inner surface of an outer layer or an outer surface of an inner layer of graft material. In yet additional embodiments, a semi-solid or solid layer of super-absorbent material can be formed and inserted between the two layers of graft material. The layer of super-absorbent material may be attached to the graft material via sutures or a biocompatible glue, or may be left unattached. Tubular body 702 includes a first graft layer 712 and a second graft layer 714 coaxial to first graft layer 712 with a space 716 therebetween. Super-absorbent material 710 is positioned inside space 716. In the embodiment shown herein, at first end 704 and/or second end 706, at least a portion of super-absorbent material 710 is exposed. In this way, when graft 700 comes into contact with blood or other liquids, super-absorbent material 710 is in contact with the liquid and is configured to swell. In some embodiments, either first end 704 or second end 706 has a connecting element 718 which connects first graft layer 712 to second graft layer 714. Connecting element 718 may be comprised of the same material as first and second graft layers 712 and 714 and may be, for example, a continuous piece of material. Alternatively, connecting element 718 may be a separate piece of material or thread which is attached to each of first and second graft layers 712 and 714. Connecting element 718 should be loosely configured, or expandable, such that upon swelling of super-absorbent material 710, first and second graft layers 712 and 714 may move apart from one another, as shown in FIG. 15, without disconnecting. In an alternative embodiment, super-absorbent material 710 is not directly exposed, and the blood or other liquids comes into contact with super-absorbent material 710 by penetration through the graft material. Penetration may occur due to diffusion, or by introduction of holes or pores in the graft material, for example.

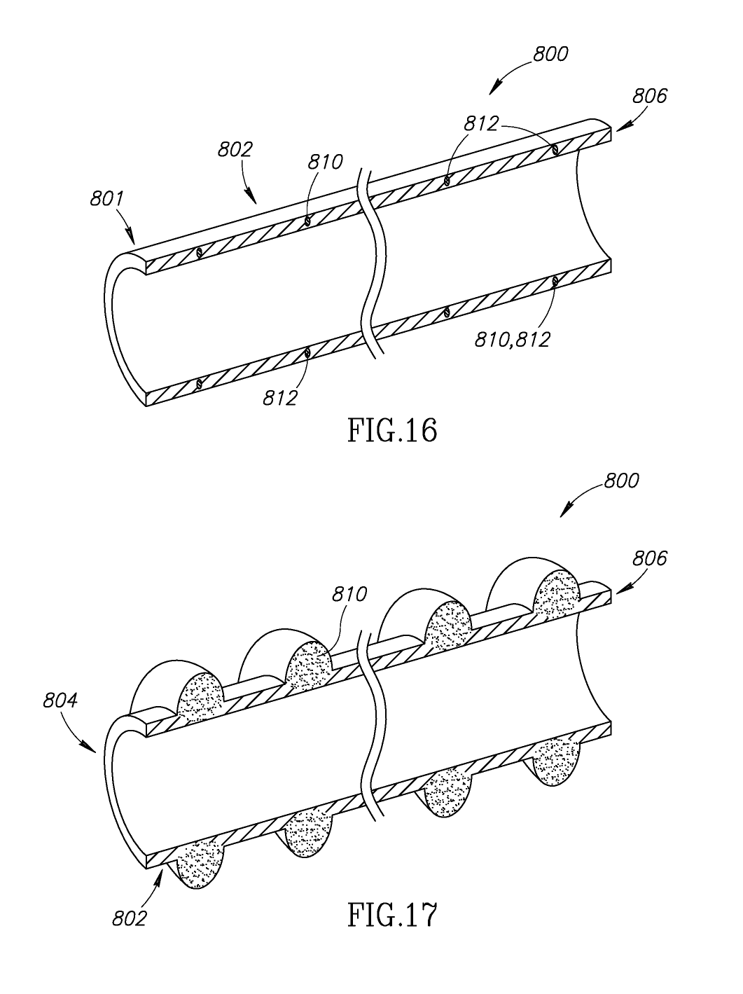

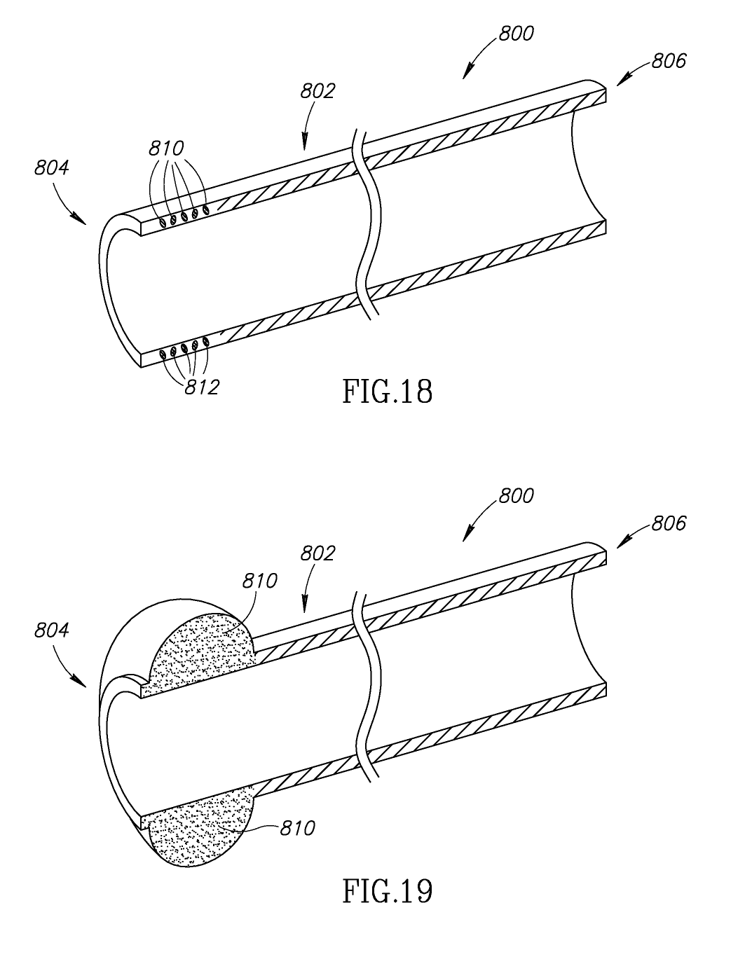

Reference is now made to FIG. 16, which is a perspective sectional view of an alternative exemplary graft 800 in an initially compressed configuration and to FIG. 17, which is a perspective sectional view of graft 800 of FIG. 16 in an expanded configuration. Graft 800 has a substantially tubular body 802 with a first end 804 and an opposing second end 806. Tubular body 802 is formed from a substantially flexible graft material. Graft 800 further includes a super-absorbent material 810 within tubular body 802. The super-absorbent material 810 may be integrated within tubular body 802. In the embodiment shown in FIGS. 16 and 17, super-absorbent material 810 includes at least one super-absorbent fiber 812 sewn into the fabric of the substantially flexible graft material. In alternative embodiments, multiple super-absorbent fibers 812 may be incorporated or sewn into the substantially flexible graft material. Super-absorbent fibers 812 may be of any suitable length. For example, in some embodiments, relatively short fibers may be used while in other embodiments, long strands may be used, which can extend along a length of tubular body 802, for example. The multiple super-absorbent fibers 812 may be spread out throughout tubular body 802, or may be concentrated in one or more areas. For example, as shown in FIGS. 16 and 17, multiple portions of tubular body 802 are configured to swell. Alternatively, first and/or second end 804 and 806 may include a high concentration of super-absorbent fibers 812 so as to form a cuff for expansion. An example of a cuff formed from a high concentration of super-absorbent fibers 812 is shown in FIGS. 18 and 19, in an unexpanded and expanded configuration, respectively. Alternatively, other portions of tubular body 802 may have a high concentration of super-absorbent fibers 812. Super-absorbent fibers 812 may be added to the graft material after manufacture, or may be sewn or knitted into the graft material during manufacture of the graft material.

In additional embodiments, super-absorbent material may be placed around or attached to tubular body 702 or 708. For example, a single long strand of super-absorbent fiber may be wrapped around the graft either in a circular pattern or a spiral pattern or any other suitable configuration.

In additional embodiments, a combination graft may include a tubular body having two layers, wherein at least one of the two layers has one or multiple super-absorbent fibers incorporated or sewn into the fabric of the layer, and may further include additional super-absorbent material in the space between the two layers. In this way, overall expansion may be accomplished together with specific/targeted expansion due to the fibers—such as at one or both ends of the graft.

In yet additional embodiments, a stent may be included so as to form a stent graft, as depicted in FIGS. 12 and 13, for example.

In one embodiment, a method is provided for treating a deformity in a blood vessel wall with graft 700 or 800, with or without a stent. Graft 700, 800 is introduced through an access site. Graft 700, 800 defines a first end and an opposing second end and includes super-absorbent material having an initial dry volume and capable of expanding to a swollen volume which is at least two times the initial dry volume. Graft 700, 800 is advanced through the blood vessel until at least a portion of graft 700, 800 extends across the deformity. With graft 700, 800 properly positioned within the blood vessel, super-absorbent material 710 or 810 is configured to expand to form a seal between graft 700, 800 and the blood vessel wall. In a particular embodiment, super-absorbent material 710, 810 is configured to harden upon expansion to facilitate retaining graft 700, 800 properly positioned within the blood vessel and/or to facilitate preventing endoleaks from forming. In some embodiments, graft 700, 800 is initially placed in a removable sheath, and the exposing is done by removing the removable sheath. In some embodiments, the exposing is done in stages, such that a first portion of the super-absorbent material is exposed and expands initially so as to anchor the graft in the vessel, and a second portion of the super-absorbent material is exposed and expands subsequent to the first portion. This allows for stable positioning of the graft within the vessel.

Although stent grafts are described hereafter, it is to be understood that grafts could utilize the same technology without being attached to a stent. While the invention has been described in terms of various specific embodiments, those skilled in the art will recognize that the invention can be practiced with modification within the spirit and scope of the claims.

1. A graft comprising:
a substantially tubular body comprising a first end and an opposing second end, said tubular body comprised of a substantially flexible graft material; and
a super-absorbent material within said substantially tubular body, said super-absorbent material having an initial dry volume, said super-absorbent material configured to absorb moisture so as to form a swollen material, said swollen material having a volume of at least twice said initial dry volume.

2. The graft of claim 1, wherein said super-absorbent material comprises at least one of: a hydrogel, a super-absorbent polymer and a super-absorbent textile.

3. The graft of claim 1, wherein said super-absorbent material comprises at least one super-absorbent fiber integrated into said substantially flexible graft material.

4. The graft of claim 3, wherein said at least one super-absorbent fiber comprises multiple super-absorbent fibers.

5. The graft of claim 4, wherein said multiple super-absorbent fibers are spread throughout said graft material.

6. The graft of claim 1, wherein said substantially flexible graft material comprises said super-absorbent material.

7. The graft of claim 1, further comprising a cuff at said first end, said cuff comprising said super-absorbent material.

8. The graft of claim 1, wherein said substantially tubular body comprises two layers of said substantially flexible graft material.

9. The graft of claim 8, wherein each of said two layers comprises at least one super-absorbent fiber having an initial dry volume, said at least one super-absorbent fiber configured to absorb moisture so as to form a swollen fiber, said swollen fiber having a volume of at least twice said initial dry volume.

10. The graft of claim 7, wherein expandable material is placed between said two layers.

11. The graft of claim 10, wherein said expandable material comprises a hydrogel.

12. The graft of claim 8, wherein said expandable material is said super-absorbent material.

13. The graft of claim 11, wherein said hydrogel material comprises one of: a powder material, a gel material, a foam material and at least one fiber.

14. The graft of claim 1, further comprising a first cuff positioned at said first end, said first cuff comprising said super-absorbent material and configured to expand and exert a radially outward force against a wall of a blood vessel.

15. The graft of claim 14, wherein said first cuff is configured to harden upon exposure to blood after said first cuff has expanded.

16. A stent graft comprising:
said graft of claim 1; and
a stent positioned with respect to said graft, said stent comprising a support structure to facilitate retaining said stent graft with respect to a deformity.

17. A method for treating a deformity in a blood vessel wall, said method comprising:
introducing a graft through an access site, the graft comprising a super-absorbent material, said super-absorbent material having an initial dry volume and capable of expanding to a swollen volume which is at least two times said initial dry volume;
advancing the graft until at least a portion of the graft extends across the deformity; and
exposing said super-absorbent material to moisture, thereby expanding said super-absorbent material to said swollen volume and filling a cross-sectional area between said graft and the blood vessel wall.

18. The method of claim 17, wherein said exposing comprises removing an outer sheath.

19. The method of claim 17, wherein said exposing is done in stages, such that a first portion of said super-absorbent material is exposed and expands initially so as to anchor said graft in the vessel, and a second portion of said super-absorbent material is exposed and expands subsequent to said first portion.

20. A graft, comprising:
a first tube of a first length and having first and second ends, the first tube comprising a first material;
a second tube, of a second length and having first and second ends and comprising a second material, coaxially disposed within the first tube and defining a first space between the first and second tubes; and
a third material disposed in the first space between the first and second tubes.

21. The graft of claim 20, wherein the third material comprises:
super-absorbent material having an initial dry volume, said super-absorbent material configured to absorb moisture so as to form a swollen material, said swollen material having a volume of at least twice said initial dry volume.

22. The graft of claim 21, wherein the super-absorbent material is exposed at at least one of the first and second ends of the first and second tubes.

23. The graft of claim 20, wherein at least one of the first and second materials comprises substantially flexible graft material.

24. The graft of claim 20, further comprising:
a connecting element coupled to each of the first and second tubes.
